(19) United States
(12) Patent Application Publication
(10) Pub. No.: US 2005/0233361 A1
(43) Pub. Date: Oct. 20, 2005

(54) PPARdelta assay

(76) Inventors: Roger G. Clerc, Binningen (CH); Martin Ebeling, Grenzach-Wyhlen (DE); Christophe Gardes, Bartenheim (FR); Markus Meyer, Neuenburg (DE); Matthew Blake Wright, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/093,816

(22) Filed: Mar. 30, 2005

(30) Foreign Application Priority Data
Mar. 30, 2004 (EP) 04101305.3
Dec. 9, 2004 (EP) 04106445.2
Jan. 27, 2005 (EP) 05100513.0

(51) Int. Cl. C12Q 1/68

(52) U.S. Cl. 435/6

(57) ABSTRACT

The present invention relates to ANGPTL4 as biomarkers for PPARdelta activity, and to methods of diagnosing a disease linked to dysregulation of PPARdelta activity such as for example dyslipidemia, obesity or insulin resistance, methods of monitoring the treatment of patients suffering from a disease linked to dysregulation of PPARdelta activity and methods of identifying compounds which modulate PPARdelta activity.

### BACKGROUND OF THE INVENTION

Peroxisome Proliferator Activated Receptors (PPARs) are members of the nuclear hormone receptor superfamily. The PPARs are ligand-activated transcription factors that regulate gene expression and control multiple metabolic pathways. Three subtypes have been described which are PPARα, PPARδ (also known as PPARβ), and PPARγ. PPARδ is ubiquitously expressed. PPARα is predominantly expressed in the liver, kidney and heart. There are at least two major isoforms of PPARγ. PPARγ1 is expressed in most tissues, and the longer isoform, PPARγ2 is almost exclusively expressed in adipose tissue. The PPARs modulate a variety of physiological responses including regulation of glucose- and lipid-homeostasis and metabolism, energy balance, cell differentiation, inflammation and cardiovascular events.

Approximately half of all patients with coronary artery disease have low concentrations of plasma High Density Lipidprotein Cholesterol (HDL-C). The atheroprotective function of HDL was first highlighted almost 25 years ago and stimulated exploration of the genetic and environmental factors that influence HDL levels. The protective function of HDL comes from its role in a process termed reverse cholesterol transport. HDL mediates the removal of cholesterol from cells in peripheral tissues including those in the atherosclerotic lesions of the arterial wall. HDL then delivers its cholesterol to the liver and sterol-metabolizing organs for conversion to bile and elimination. Data from the Framingham study showed that HDL-C levels are predictive of coronary artery disease risk independently of LDL-C (Low Density Lipidprotein Cholesterol) levels (Gordon et al., Am. J. Med. 1977, 62, 707-714). The estimated age-adjusted prevalence among Americans age 20 and older who have HDL-C of less than 35 mg/dl is 16% (males) and 5.7% (females). A substantial increase of HDL-C is currently achieved by treatment with niacin in various formulations. However, the substantial side-effects limit the therapeutic potential of this approach.

As many as 90% of the 14 million diagnosed type 2 diabetic patients in the US are overweight or obese, and a high proportion of type 2 diabetic patients have abnormal concentrations of lipoproteins. The prevalence of total cholesterol >240 mg/dl is 37% in diabetic men and 44% in women. The respective rates for LDL-C>160 mg/dl are 31% and 44%, respectively, and for HDL-C<35 mg/dl 28% and 11%, respectively. Diabetes is a disease in which a patient's ability to control glucose levels in blood is decreased because of partial impairment in response to the action of insulin. Type II diabetes (T2D) is also called non-insulin dependent diabetes mellitus (NIDDM) and afflicts 80-90% of all diabetic patients in developed countries. In T2D, the pancreatic Islets of Langerhans continue to produce insulin. However, the target organs for insulin action, mainly muscle, liver and adipose tissue, exhibit a profound resistance to insulin stimulation. The body continues to compensate by producing unphysiologically high levels of insulin, which ultimately decreases in later stage of disease, due to exhaustion and failure of pancreatic insulin-producing capacity. Thus T2D is a cardiovascular-metabolic syndrome associated with multiple comorbidities including insulin resistance, dyslipidemia, hypertension, endothelial dysfunction and inflammatory atherosclerosis.

First line treatment for dyslipidemia and diabetes generally involves a low-fat and low-glucose diet, exercise and weight loss. However, compliance can be moderate, and as the disease progresses, treatment of the various metabolic deficiencies becomes necessary with e.g. lipid-modulating agents such as statins and fibrates for dyslipidemia and hypoglycemic drugs, e.g. sulfonylureas or metformin for insulin resistance. A promising new class of drugs has recently been introduced that resensitizes patients to their own insulin (insulin sensitizers), thereby restoring blood glucose and triglyceride levels to normal, and in many cases, obviating or reducing the requirement for exogenous insulin. Pioglitazone (Actos™) and rosiglitazone (Avandia™) belong to the thiazolidinedione (TZD) class of PPARγ-agonists and were the first in their class to be approved for NIDDM in several countries. These compounds, however, suffer from side effects, including rare but severe liver toxicity (as seen with troglitazone). They also increase body weight in patients. Therefore, new, more efficacious drugs with greater safety and lower incidence of side effects are urgently needed. Recent studies provide evidence that agonism of PPARδ would result in compounds with enhanced therapeutic potential, i.e. such compounds should improve the lipid profile, with a superior effect on HDL-C rising compared to current treatments and with additional positive effects on normalization of insulin-levels (Oliver et al; Proc Nat Acad Sci USA 2001; 98: 5306-11). Recent observations also suggest that there is an independent PPARα mediated effect on insulin-sensitization in addition to its well known role in reducing triglycerides (Guerre-Millo et al; J Biol Chem 2000; 275: 16638-16642). Thus, selective PPARδ agonists or PPARδ agonists with additional PPARa activity may show superior therapeutic efficacy without the side-effects such as the weight gain seen with PPARy agonists.

### BRIEF DESCRIPTION OF THE DRAWINGS

FIG. 1: Graphical representation of ANGPTL4 gene expression fold changes induced by PPAR ligands in Table 1 (A 300 μM, B 100 nM, C 500 nM) in C2C12 mouse muscle cells measured by Affymetrix micro-arrays. ANGPTL4: Angiopoietin-like proetin [marker for PPARdelta activity], A: 2-[4-(4-Chlorobenzoyl)-phenoxy]-2-methylpropanoic acid (fenofibric acid) [PPARalpha agonist], B: {2-Methyl-4-[4-Methyl-2-(4-trifluoromethyl-phenyl)-thiazol-5-ylmethylsulfanyl]-phenoxy}-acetic acid [PPARdelta-alpha co-agonist], C: [rac]-(4-{Cyclopentyl-[4-methyl-2-(4-trifluoromethyl-penyl)-thiazol-5-yl]-methylsulfanyl}-naphtalen-1-yloxy}-acetic [PPAR delta agonist]

FIG. 2: Graphical representation form ANGPTL4 gene expression fold changes induced by PPAR ligands in Table 3 (A 300 μM, B 100 nM, C 500 nM, D 500 nM) in C2C12 mouse muscle cells measured by qPCR. ANGPTL4: Angiopoetin-like protein 4 [marker for PPARdelta activity], A: 2-[4-(4-Chlorobenzoyl)-phenoxy]-2-methylpropanoic acid (fenofibric acid) [PPARalpha agonist], B: {2-Methyl-4-[4-Methyl-2-(4-trifluoromethyl-phenyl)-thiazol-5-ylmethylsulfanyl]-phenoxy}-acetic acid [PPARdelta-alpha co-agonist], C: [rac]-(4-{Cyclopentyl-[4-methyl-2-(4-trifluoromethyl-penyl)-thiazol-5-yl]-methylsulfanyl}-naphtalen-1-yloxy}-acetic [PPARdelta agonist], D: (2-Methyl-4-{Methyl-[4-methyl-2-(4-trifluoromethyl-phenyl)-thiazol-5-ylmethyl]-amino}-phenoxy)-acetic acid [PPAR delta-alpha co-agonist].

FIG. 3A: Dotplot indicating nucleic acid conservation between the nucleotide sequence of the human and mouse ANGPTL 4 gene loci. E1 to 7 stands for ANGTPL4 gene exons one through seven. Intron 3 is indicated by a solid line, and the conserved ANGTPL4 control region is indicated by a double headed arrow.

FIG. 3B: Alignment of the 4 peroxisome proliferator response elements (PPREs) from the human (h), mouse (m), rat (r), and dog (d) ANGPTL4 genes, showing the core DR1 elements (fat) and conserved flanking sequences compared to a consensus DR1 element. The PPAR response elements located in intron 3 of the ANGPTL4 gene identified by cross-species comparison to pinpoint conserved regulatory regions. All elements are located roughly evenly spaced in an approximately 500 nucleotide region that is highly conserved across all species. Coordinates specified refer to the following genome drafts and chromosome accession numbers: human: NCBI genome build 35.1; Jun. 18, 2004/Genbank Seq ID: NT_{—}077812; mouse: NCBI genome build 33.1; Jun. 18, 2004/Genbank Seq ID: NT_{—}039649; rat: NCBI; genome build 2.1; Jul. 22, 2004/Genbank Seq ID: NW_{—}04773; dog: NCBI genome build 1.1; Aug. 30, 2004/Genbank Seq ID: NW_{—}139889.

FIG. 4: The ANGTPL4 control regions from mouse and human (SEQ IDs: 16, 17) were operably linked in a vector to a nucleic acid sequence encoding the firefly luciferase reporter gene (luciferase/luc). The resulting reporter vectors were transfected along with a PPARdelta expression plasmid into host cells. The transfected host was treated with 100 nM PPARdelta agonist {2-Methyl-4-[4-methyl-2-(4-trifluor omethyl-phenyl)-thiazol-5-ylmethyls ulfanyl]-phenoxy}-acetic acid and luciferase activity was measured. A: Fold-induction of luciferase expression in cells transfected with a PPARdelta expression vector and either human or mouse ANGPTL4 control region luciferase reporter vectors. Luciferase is expressed as fold-activation, compared to reporter-alone expressing cells. These data indicate that both mouse and human ANGPTL4 control regions are regulated by PPARdelta. B: A similar experiment in which the PPREs in the mouse reporter construct were specifically deleted by site-directed mutagenesis. Luciferase is expressed as fold-activation by agonist in cells cotransfected with both PPARdelta and luciferase reporter. The deletion of PPREs resulted in loss of transcriptional activation mediated by PPARdelta.

FIG. 5: The ANGTPL4 control region also mediates PPARgamma and PPARalpha regulation. The mouse and human ANGTPL4 control region luciferase reporter vectors were transfected into host cells along with either a PPARalpha or PPARgamma expression vector. The transfected cells were treated with 200 μM PPARalpha agonist 2-[4-(4-Chlorobenzoyl)-phenoxy]-2-methylpropanoic acid [fenofibric acid] or 100 nM PPARgamma ligand 5-[[4-[2(methyl-2-pyridinylamino)ethoxy] phenyl]-methyl]-2,4-thiazolidinedione [rosiglitazone]. Luciferase is expressed as fold-activation vs reporter-alone expressing cells.

### SUMMARY OF THE INVENTION

The present invention relates to Angiopoietin-like protein 4 as a marker for PPARdelta modulation, and methods of diagnosing a disease linked to dysregulation of PPARdelta activity, methods of monitoring the treatment of patients suffering from a disease linked to dysregulation of PPARdelta activity, and methods of identifying compounds which modulate PPARdelta activity.

### DETAILED DESCRIPTION OF THE INVENTION

It has now been discovered that the gene encoding Angiopoietin-like protein 4 (ANGL-4, ANGPTL4), also known as Fasting Induced Adipose Factor (FIAF), PPARgamma Angiopoietin Related protein (PGAR) and Hepatic Fibrinogen/Angiopoietin-Related Protein (HFARP), is a direct PPARdelta target gene and this gene may be used as a biomarker for PPARdelta.

ANGPTL4 is already described as a target gene of PPARalpha and of PPARgamma. The mRNA expression of ANGPTL4 is stimulated by PPARalpha in the liver, whereas PPARgamma stimulates a selective expression of ANGPTL4 in highly vascular tissues such as fat and placenta. This is consistent with the implication of ANGPTL4 in the metabolic response to fasting. (Kersten et al., J. Biol. Chem. 2000, 275, 28488-28493; Yoon et al., Mol. Cell. Biol. 2000, 20, 5343-5349)

However, the present invention includes the discovery that the effect of PPARdelta on the ANGPTL4 gene is in certain tissues and cells much larger than that of PPARalpha or PPARgamma. Particularly in muscle cells the ANGPTL4 gene is mainly activated by PPARdelta. Therefore, the present invention includes an assay methodology for distinguishing the response elicited by PPARdelta from the response elicited by PPARalpha.

The present invention provides the use of ANGPTL4 as marker for detecting or monitoring PPARdelta modulation. In a preferred embodiment ANGPTL4 is used as marker for detecting or monitoring PPARdelta modulation in muscle.

The term “modulation” as used herein relates to an activation or inhibition of the transcriptional activity of PPARdelta. Thus, ANGPTL4 can serve as marker for modulation of PPARdelta activity.

The term “marker” as used herein refers to a nucleic acid or polypeptide substance the measurement or determination of which can be used to indicate or quantify effects upon another biological substance, biological activity and/or disease process or syndrome.

The present invention also pertains to ANGPTL4 as marker for diagnosing a disease involving dysregulation of PPARdelta activity such as for example dyslipidemia, obesity or insulin resistance. Thus, ANGPTL4 can serve as a marker for detecting the modulation of PPARdelta activity.

The present invention further provides a method of detecting or monitoring the activity of PPARdelta in a host comprising quantifying the expression level of ANGPTL4 mRNA. A representative cDNA of mouse ANGPTL4 is shown in SEQ. ID NO: 1.

In one embodiment of the method hereinbefore described the mRNA expression level of ANGPTL4 is determined relative to a control.

A host may be an animal, tissue, cells or any other biological system that is capable of RNA transcription including in vitro transcription. Preferably, the animal is a non-human animal. The control may be the level of mRNA expression of ANGPTL4 in a different host.

Furthermore, the present invention provides a method of determining whether a test compound modulates PPARdelta activity in a host comprising exposing the host to the test compound and quantifying the mRNA expression level of ANGPTL4.

In one embodiment of the method hereinbefore described the mRNA expression level of ANGPTL4 is determined relative to a control.

A host may be an animal, tissue, cells or any other biological system that is capable of RNA transcription including in vitro transcription. Preferably, the animal is a non-human animal. The control is the level of the mRNA expression of ANGPTL4 in an untreated host, which may be the said host before the treatment or a different host, and/or the said host after an appropriate period of treatment for normalization to pretreatment levels. In a preferred embodiment of the method hereinbefore described, the compound that modulates PPARdelta activity is either an antagonist or an agonist.

The present invention also provides a method for monitoring treatment of patients suffering from a disease associated with dysregulation of PPARdelta activity such as for example dyslipidemia, obesity or insulin resistance, comprising the steps of: purifying mRNA from muscle cells isolated from patients treated with a modulator of PPARdelta activity and measuring the mRNA expression of ANGPTL4.

In one embodiment of the method hereinbefore described the mRNA expression level of ANGPTL4 is determined relative to a control.

A host may be an animal, tissue, cells or any other biological system that is capable of RNA transcription including in vitro transcription. Preferably, the animal is a non-human animal. The control is the level of the mRNA expression of ANGPTL4 in an untreated host, which may be the said host before the treatment or a different host, and/or the said host after an appropriate period of treatment for normalization to pretreatment levels.

The present invention also pertains to compounds identified by the methods hereinbefore described, and to the use of compounds identified by a method hereinbefore described for the preparation of a medicament for the treatment of a disease involving dysregulation of PPARdelta activity such as for example dyslipidemia, obesity or insulin resistance.

Several methods for measuring expression of expression levels of ANGPTL4 mRNA can be used. Methods such as Northern Blotting, and quantitation of the bands by densitometry are well known in the art and may be used, although they may not be sufficiently accurate. Other methods include the use of genechips, microarray analysis, dot blotting or different quantitative PCR methodologies. Preferably, Taqman or real time quantitative PCR is used. Any part of the transcribed sequence of ANGPTL4 may be used as probe. In a preferred embodiment, mRNA expression levels of ANGPTL4 are determined by real-time quantitative PCR using the forward primer and reverse primer listed in table 2 (SEQ. ID NOs: 4 and 5). Preferably, the mRNA expression level of ANGPTL4 in muscle cells is quantified.

The present invention further provides a method of detecting or monitoring the activity of PPARdelta in a host comprising quantifying the expression level of ANGPTL4 protein. SEQ. ID NO: 3 shows the protein sequence of mouse ANGPTL4.

In one embodiment of the method hereinbefore described the protein expression level of ANGPTL4 is determined relative to a control.

A host may be an animal, tissue, cells or any other biological system that is capable of protein translation including in vitro translation. Preferably, the animal is a non-human animal. The control is the level of the protein expression of ANGPTL4 in a different host.

Further to this the present invention provides a method of determining whether a test compound modulates PPARdelta activity in a host comprising exposing the host to the test compound and quantifying the protein expression level of ANGPTL4. In one embodiment of the method hereinbefore the protein expression level of ANGPTL4 is determined relative to a control.

A host may be an animal, tissue, cells or any other biological system that is capable of protein translation including in vitro translation. Preferably, the animal is a non-human animal. The control is the level of the protein expression of ANGPTL4 in an untreated host, which may be the said host before the treatment or a different host, and/or the said host after an appropriate period of treatment for normalization to pretreatment levels. The host may be treated with a carrier. The carrier may be a solvent in which the compound is dissolved or resuspended. In a preferred embodiment of the method hereinbefore described, the compound that modulates PPARdelta activity is either an antagonist or an agonist.

Furthermore, the present invention also provides a method for monitoring treatment of patients suffering from a disease associated with dysregulation of PPARdelta activity such as for example dyslipidemia, obesity or insulin resistance, comprising the steps of: purifying ANGPTL4 protein from total blood and/or muscle cells isolated from patients treated with a modulator of PPARdelta activity and measuring the protein expression of ANGPTL4.

In one embodiment of the method hereinbefore the protein expression level of ANGPTL4 is determined relative to a control.

A host may be an animal, tissue, cells or any other biological system that is capable of protein translation including in vitro translation. Preferably, the animal is a non-human animal. The control is the level of the protein expression of ANGPTL4 in an untreated host, which may be the said host before the treatment or a different host, and/or the said host after an appropriate period of treatment for normalization to pretreatment levels.

The present invention also pertains to compounds identified by the methods hereinbefore described, and to the use of compounds identified by a method hereinbefore described for the preparation of a medicament for the treatment of a disease involving dysregulation of PPARdelta activity.

Several methods for measuring expression levels of ANGPTL4 protein can be used. These methods include but are not limited to two-dimensional gel separation, mass-spectrometry, antibody binding techniques (ELISA, western blot) and immunoprecipitation. Preferably, the protein expression level of ANGPTL4 in muscle cells or blood is quantified.

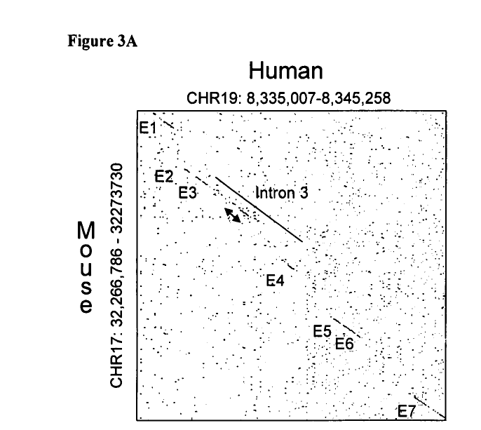

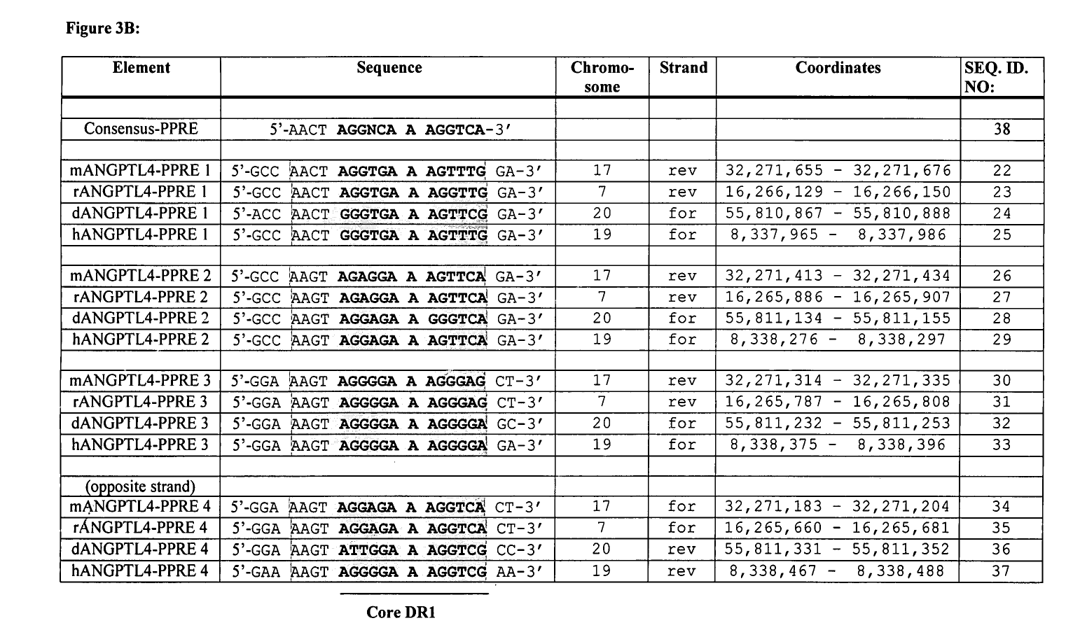

The present invention further provides a control region of FIAF/ANGPTL4 comprising binding sites for PPARs. The control region of human, mouse, rat and dog are located in the intron 3 of the corresponding ANGPTL4 gene (SEQ. ID NOs: 8-11). Preferably, the control region comprises four binding sites. The binding sites or PPAR responsive elements (PPRE's) comprise a core DR1 region of preferably 13 nucleotides (see FIG. 3). The human ANGPTL4 has four PPRE's: PPRE1 (SEQ. ID NO: 25), PPRE2 (SEQ. ID NO: 29), PPRE3 (SEQ. ID NO: 33) and PPRE4 (SEQ. ID NO: 37). The mouse ANGPTL4 has four PPRE's: PPRE1 (SEQ. ID NO: 22), PPRE2 (SEQ. ID NO: 26), PPRE3 (SEQ. ID NO: 30) and PPRE4 (SEQ. ID NO: 34). The rat ANGPTL4 has four PPRE's: PPRE1 (SEQ. ID NO: 23), PPRE2 (SEQ. ID NO: 27), PPRE3 (SEQ. ID NO: 31) and PPRE4 (SEQ. ID NO: 35). The dog ANGPTL4 has four PPRE's: PPRE1 (SEQ. ID NO: 24), PPRE2 (SEQ. ID NO: 28), PPRE3 (SEQ. ID NO: 32) and PPRE4 (SEQ. ID NO: 36). The control regions and PPRE's, respectively, of other species may be identified by methods well known in the art as for example as described in Margulies E. H. and Blanchette M. (NISC Comparative Sequencing Program, Haussler, D. and Green, E. D., Identification and Characterization of Multi-Species Conserved Sequences. Genome Res (2003), 13:2507-2518).

The present invention further provides a regulatory sequence comprising one or more sequences or fragments thereof of the group consisting of SEQ. ID NO: 22, 26, 30, 34 (mouse), or of the group consisting of SEQ. ID NOs: 23, 27, 31, 35 (rat), or of the group consisting of SEQ. ID NOs: 24, 28, 32, 36 (dog), or of the group consisting of SEQ. ID NOs: 25, 29, 33, 37 (human). Furthermore, the present invention also provides a nucleic acid comprising SEQ. ID NO: 38 or a fragment thereof. Preferably, a fragment of a PPRE comprises the 13 core DR1 region (13 nucleotides which encompass two directly repeated (DR) similar hexanucleotide segments and one intervening nucleotide). The present invention also relates to the use of the sequences SEQ. ID NOs: 22-38 as regulatory sequences.

Furthermore, the present invention provides vectors comprising at least one PPRE and a host cell comprising said vector. Preferably, the vector contains the nudeic acid of SEQ. ID. NO: 16. or 17.

Host cells can be genetically engineered (i.e., transduced, transformed or transfected) to incorporate expression systems or portion thereof for polynucleotides of the present invention. Introduction of the vector into host cells can be effected by methods described in many standard laboratory manuals, such as Davis et al., Basic methods in molecular biology Elsevier, New York (1986); Davis J M (ed.): Basic cell culture: a practical approach, sec. edition. Oxford University Press (2002); R. Ian Freshney: Culture of Animal Cells: A Manual of Basic Technique, fourth edition. John Wiley & Sons (Sd) 1999; and Sambrook et al., Molecular cloning: a laboratory manual, 2. Ed., Cold Spring Harbour Laboratory Press, Cold Spring Harbor, N.Y (1989), such as calcium phosphate transfection, DEAE-dextran mediated transfection, transvectin, microinjection, cationic lipid-mediated transfection, electroporation, transduction or infection.

A host cell may be a mammalian cell such as BHK21, HEK 293, CHO, COS, HeLa, neuronal, neuroendocrinal, neuroblastomal or glial cell lines like SH-SY5Y, PC12, HN-10, bacterial cells such as streptococci, staphylococci, E. coli, Streptomyces and Bacillus subtilis cells; fungal cells such as Saccharomyces cerevisiae and Aspergillus cell; insect cells such as Drosophila S2 and Spodoptera Sf9 cells and plant cells.

A great variety of expression systems can be used. Such a system include, among others, chromosomal, episomal and virus-derived systems, i.e. vectors derived from bacterial plasmids, from bacteriophage, from transposons, from yeast episomes, from yeast chromosomal elements, from viruses such as baculovirus, papova viruses, such as SV40, vaccinia viruses, adenovirus, fowl pox virus, pseudorabies, retroviruses and vectors derived from combinations thereof, such as those derived from plasmids and bacteriophage genetic elements, such as cosmids and phagemids. The expression systems may contain control regions that regulate as well as engender expression. Generally, any system or vector suitable to maintain, progagate or express polynucleotides to produce a polypeptide in a host may be used. The appropriate nucleotide sequence may be inserted into an expression system by any of a variety of well-known and routine techniques, such as, for example, those set forth on Sambrook et al., Molecular cloning: a laboratory manual, 2. Ed., Cold Spring Harbour Laboratory Press, Cold Spring Harbour, N.Y. (1989) or Borowski et al Trace amines: identification of a family of mammalian G protein-coupled receptors. Proc Natl Acad Sci USA (2001) 98(16):8966-71).

The present invention further provides a method for screening of modulators of PPAR activity comprising the steps of
providing a host comprising one or more PPRE's operably linked to a detectable nucleic acid, contacting said host with the candidate and
quantifying the mRNA or protein expression level of said detectable nucleic acid.

In one embodiment of the method herein before described the mRNA or protein expression level of the detectable nucleic acid is determined relative to a control.

A host may be an a non-human animal, tissue, cells or any other biological system that is capable of RNA or protein transcription including in vitro transcription and in vitro translation. The control is the level of the mRNA or protein expression of the detectable nucleic acid in an untreated host, which may be the said host before the treatment, or a different host, and/or the said host after an appropriate period of treatment for normalization to pretreatment levels. In a preferred embodiment of the method hereinbefore described, the compound that modulates PPARdelta activity is either an antagonist or an agonist.

Several methods for measuring expression levels of mRNA or protein can be used. Methods for measuring expression level of mRNA include but are not limited to methods including the use of genechips, microarray analysis, dot blotting or different quantitative PCR methodologies. Preferably, Taqman or real time quantitative PCR is used. Methods for measuring expression level of protein include but are not limited to two-dimensional gel separation, mass-spectrometry, antibody binding techniques (ELISA, western blot) and immunoprecipitation.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

### Example 1

### Microarray Experiment

### Cell culture: C2C12 cells (ATCC: CRL1772)

Cells were cultured in DMEM (5 mM glucose) supplemented with 10% heat-inactivated fetal calf serum (FCS). Complete medium was filtered through 0.2 μm pores before use. Differentiation was performed in 6 well dishes by serum depleting the medium (DMEM High Glucose, 2% FCS) when cells reach confluency. Cell cultures were then incubated for 5 days at 37° C. in a 90%-humidified and 95%-air-5%-CO₂ sterile atmosphere to allow differentiation.

PPAR Ligands were dissolved in 100% DMSO and added to medium to adjust 0.1% (v/v) DMSO final concentration and cells were incubated with ligands for 20 hours under normal growth conditions. Three replicates were done for each condition.

RNA Extraction

RNA extraction procedure has been performed with Qiagen QIAschredder and RNeasy kits.

Approximately 1*106 pelleted cells per sample were resuspended in 350 μl guanidine thiocyanate containing RLT. Samples were loaded on a QIAshredder-column and centrifuged at full speed for 2 minutes to homogenize the lysates. After adding 350 μl ethanol to the flow-through for better binding conditions, samples were applied to RNeasy spin. For the first wash step 350 μl RW1 buffer were pipeted into the column. Then 10 μL DNase solution gently mixed with 70 μl RDD buffer were applied onto the column and incubated for 15 minutes at room temperature to remove genomic DNA. DNase was washed out with 350 μl RW1 buffer. Two purifying wash steps followed by adding 500 μl RPE buffer. RNA was eluted in water and stored at −70° C.

cDNA Synthesis

cDNA was synthesized using the cDNA synthesis system kit supplied by Roche Diagnostics and following the manufacturer's manual. During the whole synthesis it was worked on ice.

For the first strand synthesis, 2 μl oligo d(T7)T₂₄ primer (5′GGCCAGTGAATTGTAATACGACTCACTATAGGGAGGCGG-(T)₂₄VN 3′, 200 pmol/μl) and redist water were added to up to 20 μg RNA for a final reaction volume of 21 μl. After incubation at 70° C. for 10 minutes in a thermal cycler and hybridization of the primers to the RNA, 2 μl AMV reverse transcriptase and 4 μl dNTP-mix (10 mM) were added. Supplementary 8 μl RT-buffer, 4 μl DTT (0.1 mM) and 1 μl RNase inhibitor were pipeted into the mixture.

Following incubation at 42° C. dured 60 minutes.

Second strand synthesis reagents were added directly into the first strand reaction tube. The second strand enzyme blend, of which 6.5 μl were applied to the mixture, contains RNase for inserting nicks into RNA of the DNA/RNA hybrid. This provides 3′OH-primers for DNA polymerase I which is also present in the 2^{nd} strand enzyme cocktail as well as E. coli ligase too. Additionally 1.5 μl dNTP-mixture, 30 μl 2^{nd} strand buffer and 72 μl redist water were added and mixed gently. The reaction mixture was incubated for 2 hours at 16° C. 20 μl added T4 polymerase ensured that the termini of the cDNA were blunt after 5 minutes incubation at 16° C. Then samples were treated with RNase to remove RNA template (30 minutes at 37° C.) and proteinase K (30 minutes, 37° C.).

Purification of cDNA

Purification using the QlAquick PCR purification kit was supplied by Quiagen.

850 μl PB buffer were added to the cDNA reaction tube and mixed. Sample were applied to the QlAquick column and centrifuged for 30 seconds at 13000 rpm. 750 μl PE buffer were added to the column to wash cDNA. An additional centrifugation step at maximum speed followed which completely dried the column. To elute DNA, 50-80 μl EB buffer (10 mM Tris-Cl, pH: 8.5) were added to the center of the membrane and the sample was centrifuged for one minute at 13000 rpm.

The cDNA quality was checked by agarose gel electrophoresis and ethidium bromide staining.

Smears from 100 to >10000 bp appeared. The quantity and purity of the synthesized cDNA was determined by measuring the absorbance at 260 nm and 280 nm in a spectrophotometer.

The concentration was calculated as follows: c[μg/ml]=A₂₆₀*50*D, (50: dsDNA specific factor, D: dilution factor). cDNA has a A₂₆₀/A₂₈₀-ratio of 1.8-2.1

In Vitro Transcription (IVT)

This step was performed using MEGAscript T7 kit from Ambion.

5 μg cDNA from each samples were used as template and mixed with ATP, GTP, CTP, UTP (Ambion) and biotinylated CTP (Bio-11-CTP, ENZO) and UTP (Bio-15-UTP, ENZO). RT was performed by T7 enzyme mix for 4 hours at 37° C. Transcription products were purified with RNeasy kit from Qiagen following the same procedure as for RNA extraction, without any DNAse treatment of the samples.

Fragmentation of IVT Products

15 μg RNA from IVT were fragmented in 200 mM Tris-Acetate, pH 8.1, 500 mM KOAc, 150 mM MgOAc for 35 minutes at 95° C. in a small volume (20 to 30 μl).

Chip Handling (cf DNA Micro-array Protocols V3-200.1. M Wilhelm-Seiler, U Certa)

A pre-treatment solution was first applied on Genechip (6.25 μl Acetylated BSA (20 μg/μl), 12.5 μl salmon sperm DNA (10 μg/μl), 125 μl 2×MES Hyb buffer, 106.25 μl H₂O). Genechip were incubated for 15 min at 40° C. with a rotation of 60 rpm in a rotisserie.

Samples were prepared for hybridization as follows: mix 15 μg fragmented RNA with 2.5 μl control stock mix which contains BioB, BioC, BioD, Cre (internal references), 2.5 μl salmon sperm DNA (10 μg/μl), 6.25 μl Acetylated BSA (20 μg/μl), 125 μl 2×MES-Hyb buffer, 91.25 μl H₂O. Microarrays were then incubated overnight at 45° C. with rotation (60 rpm) in a rotisserie.

Samples were removed and stored at −20° C. Microarrays were washed with 6 SSPE for 5 minutes in the fluidics. Then 230 μl MES-Wash buffer were applied to the chip which was incubated at 45° C. for 30 minutes, 60 rpm.

Staining was performed by incubating chips with the following solution for 15 minutes: 125 μl 2× Stain Buffer, 91.25 Acetylated BSA, 2.5 μl Streptavidin (1 mg/ml).

Staining solution was removed, micro-arrays washed with 6×SSPE for 5 minutes in the fluidics Station, and treated with the amplification solution: 125 μl 2× Stain Buffer, 99 μl H₂₀, 1 μl Biotinylated-anti-Streptavidin (500 μg/ml), 25 μl Acetylated BSA (20 μg/μl). Incubation for 30 minutes at 40° C. with 60 rpm in the rotisserie. After washing I the fluidics Station with 6×SSPE, micro-arrays were incubated with phycoerytrin solution (125 μl 2× Stain buffer, 91.25 μl H₂O, 31.25 μl acetylated BSA, 2.5 μl Phycoerytrin (1 mg/ml)) at 40° C., with 60 rpm.

Solutions:

12×MES stock: 70.4 g MES free acid monohydrate, 193.3 g MES sodium salt, adjust pH to 6.6, ad 1000 ml with tridest H₂O
2×MES-Hyb Buffer: 8.3 ml 12×MES stock, 17.7 ml 5M NaCl, 4 ml 0.5M EDTA, 0.1 ml 10% Tween 20, 19.9 ml Tridest H₂O
MES-Wash Buffer: 83.3 ml 12×MES stock, 5.2 ml 5M NaCl, 1 ml 10% Tween 20, ad 1000 ml with tridest H₂O
6×SSPE Tween: 300 ml 20×SSPE, 1 ml 10% Tween 20, ad 1000 ml tridest H₂O
2× Stain Buffer: 41.7 ml 12×MES stock, 92.5 ml 5M NaCl, 2.5 ml 10% Tween 20, 113.3 ml tridest H₂O

Scanning is performed with Affymetrix scanner and results were provided on Gene Chip software from Affymetrix.

Chip Data Analysis

Data were analyzed using Race A Sofware (Bioinformatic Roche). Triplicates were used for each condition and comparisons were calculated for each condition referring to the control (untreated cell samples). Several criteria have been taken into account for the analysis of each gene: pValue, Call average, Sum average difference, Change Factor and dispersion in each condition to get a list of the most statistically relevant genes that are modulated by the studied compounds.

Results

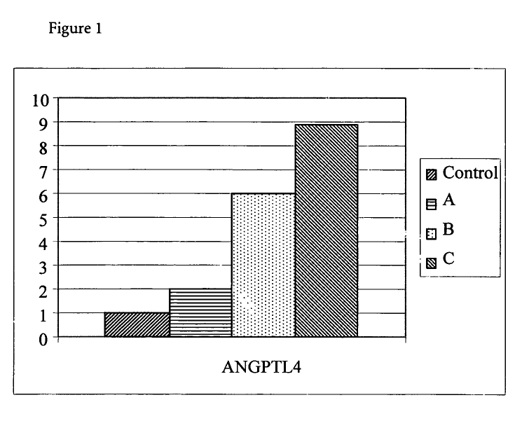

A novel selective PPAR delta biomarker gene was identified from Affymetrix micro-array experiments (see FIG. 1): ANGPTL4 (angiopoetin-like protein 4) (SEQ. ID NO: 2). This marker gene allows to differentiate PPAR alpha from PPAR delta activities.

The tested ligands were a PPARalpha agonist (A: 2-[4-(4-Chlorobenzoyl)-phenoxy]-2-methylpropanoic acid (fenofibric acid)), a PPARdelta agonist (C: [rac]-(4-{Cyclopentyl-[4-methyl-2-(4-trifluoromethyl-penyl)-thiazol-5-yl]-methylsulfanyl}-naphtalen-1-yloxy}-acetic) and a PPARdelta-alpha co-agonist (B: {2-Methyl-4-[4-Methyl-2-(4-trifluoromethyl-phenyl)-thiazol-5-ylmethylsulfanyl]-phenoxy}-acetic acid).

As shown in Table 1 and FIG. 1 the response of the marker gene ANGPTL4 elicit by the PPARdelta agonist (C) and the PPARdelta-alpha co-agonist (B) is far larger than the response elicited by the PPARalpha specific ligand (A).

TABLE 1
ANGPTL4 gene expression fold changes in C2C12 mouse muscle cells
induced by PPAR ligands: A 300 μM, B 100 nM, C 500 nM measured
by Affymetrix micro-arrays. (A: 2-[4-(4-Chlorobenzoyl)-phenoxy]-2-
methylpropanoic acid (fenofibric acid) is a PPARalpha agonist,
B: {2-Methyl-4-[4-Methyl-2-(4-trifluoromethyl-phenyl)-thiazol-5-
ylmethylsulfanyl]-phenoxy}-acetic acid is a PPARdelta-alpha co-agonist,
C: [rac]-(4-{Cyclopentyl-[4-methyl-2-(4-trifluoromethyl-penyl)-thiazol-5-
yl]-methylsulfanyl}-naphtalen-1-yloxy}-acetic is a PPAR delta agonist)

| | Symbol | Control | A | B | C |
|---|---|---|---|---|---|
| Angiopoetin-like protein | ANGPTL4 | 1.00 | 1.99 | 6.00 | 8.87 |

### Example 2

### Quantitative PCR

The culturing of the C2C12 mouse muscle cells, RNA extraction, cDNA synthesis and purifications were performed as described in Example 1.

PCR Primer Design

Primer pairs were designed with the software Primer express 1.0 from PE Applied Biosystems based on mRNA/cDNA sequences from Medline. Criteria for each primer were length between 19 to 21 nucleotides, melting temperature of 60° C.+/−1, G/C content range from 40 to 60% and avoidance of secondary structures as hairpins or primer dimers. Additionally the length of the amplicon was controlled as shorter amplicons amplify more efficiently than longer ones and are more tolerant of reaction conditions (usually ˜100 bp). Primers were dissolved in DEPC treated water (Ambion) to a 100 μM concentrated solution and stored at −20° C.

TABLE 2
Forward and reverse primers of gene S12 (internal reference) and ANGPTL4.

| | | | | SEQ. | | | SEQ. |
|---|---|---|---|---|---|---|---|
| GENE | ACC | OLIGO NAME | FORWARD PRIMER | ID: | OLIGO NAME | REVERSE PRIMER | ID: |
| S12 | Y11682 | mS12_98F | TGAACCAGATGCACCGCTTAG | 6 | mS12_416R | TTCTTCTTTTGCACGTGGCC | 7 |
| ANGPTL4 | AF278699 | mANGL4_893F | TGCTCCAATTTCCCATCCAAT | 4 | mANGL4_952R | CAGTGAGCTGCAGGCTGTAGG | 5 |

qPCR using Corbett RG3000 and ABI 7000

RT-PCR was performed with the Quantitech SYBR Green kit from Qiagen according to the manufacturer's manual. Each component were added to 96 well plate: 25 μl master mix, 0.16 μl of each primer (100 μM stock solution), 22.7 μl H₂O and 2 μl cDNA.

The fluorescent dye SYBR green I binds the minor groove of double stranded DNA and allows measurement of DNA quantity at every cycle. The program consists of three different steps: initial denaturation of template and heat-activation of the Taq DNA polymerase (95° C., 15 min), cycling (95° C. denaturation, 60° C. primer-template annealing, 72° C. elongation, fluorescence aquisition), and final melting (melting curve 55 to 95° C., measurements in 0.5° C. steps). The melting curve allows the discrimination between specific and non-specific amplification products (primer dimers).

Relative Quantification Using Real-Time PCR

The relative quantification is based on the relative expression of the target gene versus a reference gene. The concept of threshold fluorescence, defined as the point where fluorescence rises above the background fluorescence enables accurate and reproducible quantification of gene expression. The cycle number at which threshold fluorescence is reached, is called ct value. A linear relation between ct value and the log of the number of initial molecules is given during the exponential phase of the amplification reaction. Therefore, quantification is reliable at that stage and will not be affected by any limiting effects of the components. The relative ct value of a target gene compared to the ct value of an internal reference in the control or untreated sample is expressed as Δct.control=ct_{reference of control}−ct_{target of control} and in a treated, unknown sample as Δct.sample=ct_{reference of sample}−ct_{target of sample}.

The theoretical PCR reaction is expressed through the formula N=N₀*2ⁿ (N: number of amplified molecules, N₀: initial number of molecules, n: number of amplification cycles) where the base 2 represents the optimal efficiency (one doubling every cycle). This equation allows to illustrate differential expression in mRNA copy number instead of ct values: the exponential value with the theoretical efficiency, 2^{Δct}, is taken.

As internal reference for quantitative studies the ribosomal protein S12 gene expression was used. It is expressed constitutively and at the same level in all samples. Setting of fluorescence threshold is determined by the light cycler software. The obtained ct values (duplicates for one sample, two samples per condition) were exported into Microsoft Excel and analyzed as described above.

Results

The novel selective PPAR delta biomarker gene (SEQ. ID. NO: 2) identified from Affymetrix micro-array experiments (see FIG. 1) was confirmed by qPCR (quantitative polymerase chain reaction).

The tested ligands were a PPARalpha agonist (A: 2-[4-(4-Chlorobenzoyl)-phenoxy]-2-methylpropanoic acid (fenofibric acid)), a PPARdelta agonist (C: rac]-(4-{Cyclopentyl-[4-methyl-2-(4-trifluoromethyl-penyl)-thiazol-5-yl]-methylsulfanyl}-naphtalen-1-yloxy}-acetic) and two PPARdelta-alpha co-agonists (B: {2-Methyl-4-[4-Methyl-2-(4-trifluoromethyl-phenyl)-thiazol-5-ylmethylsulfanyl]-phenoxy}-acetic acid and D: (2-Methyl-4-{Methyl-[4-methyl-2-(4-trifluoromethyl-phenyl)-thiazol-5-ylmethyl]-amino}-phenoxy)-acetic acid).

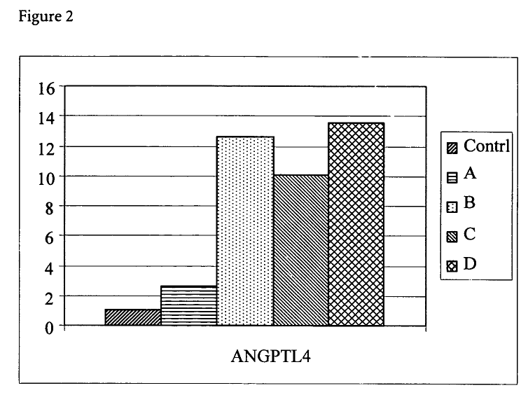

As shown in Table 3 and FIG. 2 the response of the marker gene ANGPTL4 elicited by the PPARdelta agonist (C) and PPARdelta-alpha co-agonists (B and D) is far more larger than the response elicited by the PPARα specific ligand (A).

TABLE 3
ANGPTL4 gene expression fold changes in C2C12 mouse muscle cells
induced by PPAR ligands in C2C12: A 300 μM, B 100 nM, C 500 nM,
D 500 nM as measured by qPCR. (A: 2-[4-(4-Chlorobenzoyl)-phenoxy]-2-
methylpropanoic acid (fenofibric acid) is a PPARalpha agonist,
B: {2-Methyl-4-[4-Methyl-2-(4-trifluoromethyl-phenyl)-thiazol-5-
ylmethylsulfanyl]-phenoxy}-acetic acid is a PPARdelta-alpha co-agonist,
C: [rac]-(4-{Cyclopentyl-[4-methyl-2-(4-trifluoromethyl-penyl)-thiazol-5-
yl]-methylsulfanyl}-naphtalen-1-yloxy}-acetic is a PPARdelta agonist,
D: (2-Methyl-4-{Methyl-[4-methyl-2-(4-trifluoromethyl-phenyl)-thiazol-5-
ylmethyl]-amino}-phenoxy)-acetic acid is a PPARdelta-alpha co-agonist)

| | Symbol | Control | A | B | C | D |
|---|---|---|---|---|---|---|
| Angiopoetin-like | ANGPTL4 | 1 | 2.61 | 12.58 | 10.06 | 13.51 |
| protein | | | | | | |

Specific PPARdelta ligands such as GW501516 are able to elicit many biological responses similar to the effects of well-known PPARalpha ligands (fibrates) both in vitro and in vivo. One major biological difference is differential regulation of ANGPTL4 expression as demonstrated by oligonudeotide arrays and quantitative PCR. ANGPTL4, a newly discovered circulating cytokine, is regulated by fasting in liver, adipocytes and muscle; it has recently been demonstrated to be a direct PPAR target gene and to be associated with lipolysis. Hence ANGPTL may provide a new approach to i. selectively distinguish the roles of PPARdelta from PPARalpha and ii. to develop more differentiated medicines for dyslipidemia and type 2 diabetes.

### Example 3

### Comparative Genomic Analysis

The human (NCBI genome build 35.1; Jun. 18, 2004), rat (NCBI; genome build 2.1; Jul. 22, 2004), mouse (NCBI genome build 33.1; Jun. 18, 2004) and dog (NCBI genome build 1.1; Aug. 30, 2004) ANGPTL4 genes were compared. First, BLAST comparisons were perfomed to provide convenient “anchor points” for more sensitive in depth analysis of orthologous genomic regions. Then, conserved genomic regions were further analyzed using homology-based gene predictions. For the ANGPTL4 gene, various conserved stretches within the gene's introns were found as illustrated by a dot plot (FIG. 3A) comparing the genomic regions of man and mouse. The best-conserved stretch is located within intron 3. It was readily detected by a BLAST search and it is even more highly conserved than the protein encoding exons of the ANGPTL4 gene.

To further elucidate the possible relevance of these conserved intronic stretches, known transcription factor binding sites (TFBSs) were mapped. All TFBSs from TransFac8.1 [BIOBASE (biological databases), Halchtersche Strage 33, D-38304 Wolfenbuüttel, Germany] for which a matrix-based description was available were included. While the mapping algorithm used adjusts for statistical significance of TFBS matches, about five TFBS predictions are still obtained for any arbitrary genomic sequence stretch of 100 bp. However, based on the assumption that both regulatory mechanisms and consensus TFBSs tend to be conserved among the higher vertebrates, one can combine the TFBS predictions with alignments of the corresponding orthologous genomic regions, and with conservation profiles derived from those alignments. This allows those TFBS predictions that do not appear to be sufficiently well conserved to be excluded.

Results

In the conserved stretch of the ANGPTL4 gene's intron 3, about 50 matches to TFBSs in TransFac8.1, including several matches to peroxisome proliferator activated receptor response element motifs (PPRE's/DR1) were identified. All four of the PPRE-related matches coincided with the four highest peaks in the conservation plot. None of the other predicted TFBSs overlapped with any of the conservation peaks. Moreover, there were no additional PPREs predicted outside of the most conserved regions. At the nucleotide level, all of the putative PPREs are well-conserved among the four species, and are excellent matches to the consensus PPRE/DR1 element (FIG. 3B). Thus, the computational analyses identified four separate PPREs in intron 3 of ANGPTL4, of which three are on the forward (coding) strand and one on the reverse strand.

### Example 4

### PCR Amplification of Mouse and Human ANGPTL4 Control Region

PCR primer pairs were designed to amplify a segment of ANGPTL4 gene intron 3 from mouse and human genomic DNA encompassing the 4 putative PPREs. Primers were designed with the software Primer Express 2.0 from PE Applied Biosystems (Headquater: 850 Lincoln Centre Drive, Foster City, Calif. 94404, USA) using similar parameters as described in Example 2. PCR amplification was performed using the pfu PCR mix from Stratagene. Each reaction used 50 ng of template DNA, 10 pMoles each of both forward and reverse primers, with recommended amount of dNTPs, 10× buffer, and PCR enzyme in a final reaction volume of 50 μl.

TABLE 4
Forward and reverse primer sequences for PCR cloning of ANGPTL4 control region

| | | | SEQ. | | | SEQ. | Product |
|---|---|---|---|---|---|---|---|
| TARGET | Oligo name | Forward primer | ID | Oligo name | Reverse primer | ID | length |
| mANGPTL | mANGPTL4clonF2 | CGTTCACCCTTCT | 12 | mANGPTL4clonR2 | CTTGGCCTTGAC | 13 | 582 bp |
| 4 control | | TGACATCTGTG | | | AAGTCCTCTAA | | |
| region | | | | | | | |
| hANGPTL4 | hANGPTL4clonF2 | CTCGCGGTTCTCT | 14 | hANGPTL4clonR2 | AGGAGGCAGGGT | 15 | 711 bp |
| control | | AAGTTCACGG | | | TGAGGAAAGAAA | | |
| region | | | | | | | |

PCR amplification was carried out in a T3 thermocycler from Biometra (Whatman Biometra, Biometra GmbH i. L., Rudolf-Wissell-Straβe 30, 37079 Goettingen, Germany). The amplification program consisted of initial denaturation of template (95°, 2 min), 35 repetitions at 95°, 60 sec denaturation; 50-65° Gradient, 30 sec annealing; 72°, 4 min extension. Following PCR, an aliquot was separated by agarose gel electrophoresis and quantity and size of amplified bands was visualized by ethidium bromide staining.

Construction of ANGPTL4 Control Region-Luciferase Reporter Vectors

The PCR products were subcloned into a TA vector and their DNA sequences were determined. The inserted PCR products with the expected sequence were recovered by restriction digestion of the insert-containing TA plasmids and subcloned into a luciferase reporter vector containing a minimal −37 TK promoter (pGL3basic-pTK-37Luc: insertion of minimal promoter region from the herpes simplex thymidine kinase gene (pTK37) (SEQ. ID NO: 39) between BglII and EcoRI restriction sites of the pGL3-Basic vector (Promega, Aceession number: U47295)). This resulted in two plasmids; mANGPTL4controlregion-Luc and hANGPTL4controlregion-Luc in which luciferase expression is directed by the ANGPTL4 control region from mouse and human, respectively. Large amounts of these plasmids were prepared in E coli for subsequent studies.

Mutagenesis

To verify that PPARdelta transcriptional control of the ANGPTL4 control region is dependent upon the 4 identified PPREs, they were made nonfunctional individually and in combination, by site-directed mutagenesis of the mANGPTL4controlregion-Luc plasmid. Primers were produced to delete the 13 core DR1-encoding nucleotides of each PPRE. Mutagenesis was carried out using the Quikchange Multi Site Directed Mutagenesis Kit from Stratagene. Properly mutagenized vectors containing deletion(s) of one or more of the PPREs were identified by DNA sequencing. Large amounts of these plasmids were prepared in E coli: for subsequent studies.

TABLE 5

Primer sequences for mutagenesis of PPREs
in the mFIAF/ANGPTL4 control region

| | | | SEQ. ID |
|---|---|---|---|
| TARGET | OLIGO NAME | PRIMER SEQUENCE | NO: |
| mANGPTL4 | mAngptl4 del | CTCCACAGCCAACTGATATC | 18 |
| PPRE1 | PPRE1 | CCCTTCAC | |
| mANGPTL4 | mAngptl4 del | CAGCCTAGCCAAGTGAGCTG | 19 |
| PPRE2 | PPRE2 | GAGAGACA | |
| mANGPTL4 | mAngptl4 del | GATGAGAGGAAAGTCTAGCT | 20 |
| PPRE3 | PPRE3 | GCCCGAGG | |
| mANGPTL4 | mAngptl4 del | TGCCCCTCCCCCAGACTTTC | 21 |
| PPRE4 | PPRE4 | CCTGGCTG | |

Luciferase Assays

Baby hamster kidney cells (BHK21 ATCC CCL10) were grown in DMEM medium containing 10% FBS at 37° C. in a 95% O2:5% CO₂ atmosphere. Cells were seeded in 6 well plates at a density of 2×10⁵ cells/well and then batch-transfected with vectors expressing full-length human PPARδ, PPARγ or PPARα receptors along with the appropriate ANGPTL4controlregion-Luc reporter plasmid. Transfection was accomplished with the Fugene 6 reagent (Roche Molecular Biochemicals) according to the suggested protocol. Six hours following transfection, the cells were harvested by trypsinization and seeded in 96 well plates at a density of 10⁴ cells/well. After 24 hours to allow attachment of cells, the medium was removed and replaced with 100 μl of phenol red-free DMEM medium containing the test substances or control ligands (final DMSO concentration: 0.2%). Following incubation of the cells for 24 hours with substances, 50 μl of the supernatant was discarded and then 50 μl of Steady-Glo Luciferase Assay Reagent (Promega) to lyse the cells and initiate the luciferase reaction was added. Luminescence for luciferase was measured in a Packard TopCount. Transcriptional activation in the presence of a test substance was expressed as fold-activation over cells incubated in the absence of the substance.

Results

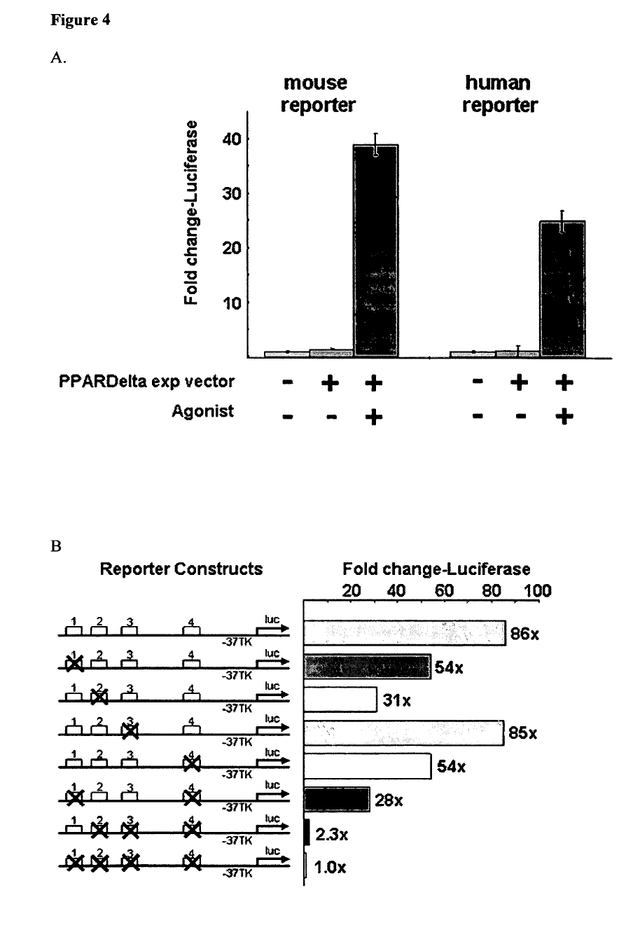

FIG. 4A shows that PPARdelta mediates transcriptional control of the heterologous luciferase reporter via the FIAF/ANGPTL4 control regions. Cells transfected with PPARdelta receptor vector along with luciferase reporter showed no increase in transcriptional activation compared to cells transfected with the reporter alone. However, after treatment with PPARdelta agonist, luciferase activity increased by 39-fold and 25-fold for the mouse and human FIAF/ANGPTL4 control region-Luc reporters, respectively. In FIG. 4B, a similar experiment was performed but using mFIAF/ANGPTL4 control region-Luc reporters in which one or more of the PPREs was deleted. Deletion of PPREs 1, 2, and 4 alone caused a decrease in PPARdelta-mediated transcription. Deletion or more than one PPRE resulted in further losses in transcriptional activation with all regulation lost upon deletion of all four PPREs. Thus PPARdelta robustly and specifically regulates transcription via the 4 PPREs contained within the mouse and human FIAF/ANGPTL4 control regions.

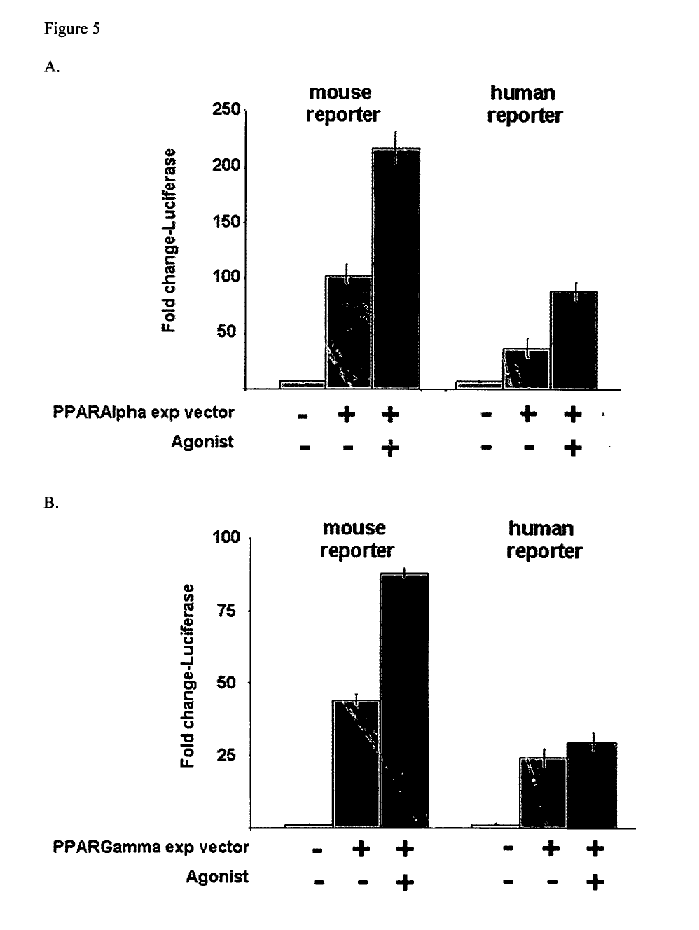

Since PPARalpha and PPARgamma agonists have been shown to induce FIAF/ANGPTL4 expression in liver and adipose tissue, respectively, they were also tested for regulation of the ANGPTL4 control region. FIG. 5 shows that both PPARalpha and PPARgamma can transcriptionally regulate the mouse and human ANGPTL4 control regions. In contrast to PPARdelta, transfection of cells with PPARalpha and PPARgamma resulted in increased activation even in the absence of agonist, that was further increased upon treatment with selective agonists.

1. A method of detecting or monitoring the activity of PPARdelta in a host comprising determining the level of ANGPTL4 mRNA or protein in a biological sample from said host.

2. The method according to claim 1 comprising determining the mRNA expression level of ANGPTL4 relative to a control.

3. A method of determining whether a test compound modulates PPARdelta activity in a host comprising
a) exposing the host to the test compound and
b) determining whether the level of ANGPTL4 mRNA or protein has changed following exposure to the test compound.

4. A method according to claim 3 comprising determining the mRNA expression level of ANGPTL4 relative to a control.

5. The method of claim 1 comprising the steps of
a) purifying RNA from muscle cells isolated from patients treated with a modulator of PPARdelta activity and
b) measuring the mRNA expression of ANGPTL4.

6. The method according to claim 5 comprising measuring the mRNA expression level of ANGPTL4 relative to a control.

7. A compound identified by the method of claim 3.

8. The method according to claim 1 comprising quantifying the protein expression level of ANGPTL4.

9. The method according to claim 8 comprising the step of determining the protein expression level of ANGPTL4 relative to a control.

10. The method according to claim 3 comprising
a) exposing the host to the test compound and
b) quantifying the protein expression level of ANGPTL4.

11. The method according to claim 10 comprising determining the protein expression level of ANGPTL4 relative to a control.

12. The method of claim 1 comprising the steps of
a) purifying protein from total blood and/or muscle cells isolated from patients treated with a modulator of PPARdelta activity and
b) measuring the protein expression of ANGPTL4.

13. The method according to claim 12 comprising determining the protein expression level of ANGPTL4 relative to a control.

14. An isolated PPAR responsive nucleic acid sequence selected from the group consisting of SEQ. ID. NOS. 22-38 and fragments thereof.

15. The nucleic acid of claim 14 selected from the group consisting of SEQ. ID NOS: 22, 26, 30, 34 and fragments thereof.

16. The nucleic acid of claim 14 selected from the group consisting of SEQ. ID NOs: 23 27, 31, 35 and fragments thereof.

17. The nudeic acid of claim 14 selected from the group consisting of SEQ. ID NOs: 24, 28, 32, 36 and fragments thereof.

18. The nucleic acid of claim 14 selected from the group consisting of SEQ. ID NOs: 25, 29, 33, 37 and fragments thereof.

19. The nucleic acid of claim 14 which is SEQ. ID NO: 38 or a fragment thereof.

20. A method for screening of modulators of PPAR activity comprising the steps of
a) providing a host cell genetically engineered to contain one or more PPAR responsive nucleic acid sequences selected from the group consisting of SEQ. ID NOS. 22-38 operably linked to a detectable nucleic acid,
b) contacting said host cell with a candidate compound and
c) quantifying the mRNA or protein expression level of said detectable nucleic acid.

21. The method according to claim 20 comprising determining the mRNA or protein expression level of the detectable nucleic acid relative to a control.


SEQUENCE LISTING (39 sequences)

SEQ ID NO: 1 - DNA, 1883 bp, Mus musculus
  Angiopoietin-like protein 4 (ANGPTL4) (1)..(1870): representative cDNA (complete cds) (BC021343 as of 25.03.2004), for gene under UniGene ID Mm.196189
ccacgcgtccggctacgggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagaggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacaactcacagcattcttacaataaaaacaacctcagaacaaaaaaaaaaaaaaa

SEQ ID NO: 2 - DNA, 1858 bp, Mus musculus
  Angiopoietin-like protein 4 (ANGPTL4) (1)..(1858): representative cDNA (NM_020581, as of 25.03.2004), for gene under UniGene ID Mm.196189
gcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgctcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctggacaatggagtagacaagacttcgaggggaaagaagctttccaagatgacccagctcattggcttgacttccaacgccacccacttacacaggccggcccgggactgccaggaactcttccaagaaggggagaggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacaggggaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgttgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacaaaacaaaaaaaaaaaaaaaaaaaa

SEQ ID NO: 3 - protein, 410 aa, Mus musculus
  Angiopoietin-like protein 4 (ANGPTL4) (1)..(410): protein sequence (NP_065606, as of 25.03.2004), of gene under UniGene ID Mm. 196189
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKKLSKMTQLIGLTSNATHLHRPARDCQELFQEGERHSGLFQIQPLGSPPFLVNCEMTSDGGWTVIQRRLNGSVDFNQSWEAYKDGFGDPQGEFWLGLEKMHSITGDRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 4 - DNA, 21 bp, Mus musculus
  ANGPTL4 forward primer (1)..(21)
tgctccaatttcccatccaat

SEQ ID NO: 5 - DNA, 21 bp, Mus musculus
  ANGPTL4 reverse primer (1)..(21)
cagtgagctgcaggctgtagg

SEQ ID NO: 6 - DNA, 21 bp, Mus musculus
  S12 forward primer (1)..(21)
tgaaccagatgcaccgcttag

SEQ ID NO: 7 - DNA, 20 bp, Mus musculus
  S12 reverse primer (1)..(20)
ttcttcttttgcacgtggcc

SEQ ID NO: 8 - DNA, 624 bp, Homo sapiens
  human ANGPTL4 intron 3 containing four PPREs (1)..(624): segment of human chromosome 19 (GenBank NT_077812), region from 8337915  to 8338538
cacggtttctctgtggtcctcatccttccctgcatctgtggctgtccacagccaactgggtgaaagtttggatccccctctcacaccctagggtcagtggcaggcttccagcactgtagacctgagggttctctcctccccaagctcccgctccttcccacctccgtgctgcccgcccccaaccccgccaggctagcatctcagcgtggtcagggtcctgtccaccctcccaaagccaccatcccaggatgaggggcttctggagggtgacgggggaaggcacaagtcctggctgggaaatgcggtggaagggggcaggggttctgtgggttcgggactcccagactcttggctcaggcccgccaagtaggagaaagttcagagctgggaaggcgaacagctggcattcatggaagccacactggtggtttggccgcgtgcccatccttactggatgggaggaaagtaggggaaaggggagatgcctgaggggccggaaagcgtcttcctggtcactctgggcccgcccccacccccaccgtgcagactcatttcgacctttcccctacttttccggctgggctgggggcggttcctcccagtctggagcgtctgagcctccag

SEQ ID NO: 9 - DNA, 594 bp, Mus musculus
  ANGPTL4 intron 3 containing four PPREs (1)..(594): Segment of chromosome 17 (GenBank NT_039649 as of 11.01.2005), region  from 32271133  to 32271726
gccttgacaagtcctctaaattcaggaagaaccacccccagcccagccagggaaagtaggagaaaggtcactgggggaggggcatgcaggcactttctgccagccagctccgtctgcactgccaggagtgggggcagacccagaaagatgggaagtatactttccaatccctcgggcagctagctccctttcccctactttcctctcatccaatcgggacaggcacactgccaaaccacccatgtggttcccattagggccagctgtttgtctctccagctctgaactttcctctacttggctaggctgagtctgggagttccaaagccacaaaaccaccccctcagctcttctcttccaccacatcacccatccagaacctatgcctgccgaccctggaaaagctcatccatgaccctcgccatgcctaggtacttgcctaagtaaaagccaggggaggaattaagtctaaggaggattgggctgaaagccagcaagcagccttagggtgtgaaggggatatccaaactttcacctagttggctgtggagagccacagatgtcaagaagggtgaacgaataggcatgaactca

SEQ ID NO: 10 - DNA, 585 bp, Canis familiaris
  dog ANGPTL4 intron 3 comprising four PPREs (1)..(585): Segment of chromosome 20 (GenBank NW_139889 as of 11.01.2005), region  from 55810817  to 55811401
cggtcctcctaccccatcacaccccagcctggcatcagtggctgcgtgcaaccaactgggtgaaagttcggattcccccctttcacaccctggggctggttgctggctgtcagccctttagtcttgagggctcctcctcccttcccctccctccaactccctcccctcctccccattcccaggccagtaagggtccagtacaccctcccaaacctcctccctgggatcagaccggcaggcgcgggtctgggtcggggatgtgggggtgcggggggggtgcgtgggtttggggactcccagacccggggctcagccctgccaagtaggagaagggtcagagctggagagacaaacagctggtgttaatggaagccacattggtggtttggcccggtgccgtggggattggaagggaggaaagtaggggaaaggggagctgctccaggggccggaagaggtctccagtcactctgggtccgcccccctccccccagcgtggacttcccctcccccgggcgacctttccaatactttccccggctgggctggcactggatgcgggctttcatcagcagtttggacgcg

SEQ ID NO: 11 - DNA, 591 bp, Rattus norvegicus
  rat ANGPTL4 intron 3 comprising 4 PPREs (1)..(591): segment of rat chromosome 7 (GenBank NW_047733 as of 11.01.2005), region from 16265610 to16266200
gccttgacaagtcctctaaattcaggaagaaccgcccccagcccagccagggaaagtaggagaaaggtcactgggggaggagcatgcagggactttctgccagccagctcagtccacactgccaggagtgggggcagacccagaaagatgggaagtctactttccaatcccttgggcagctccctttcccctactttcctctcatccaatcgggacaggcacactgccaagccacccatgtggttttcattagggccagctgtttgtctccccagctctgaactttcctctacttggctaggctgagtctgggagtcccaaacccacaaaaccaccccctcagctcttctcttccaccacatcacctatccagaagctatgcctgccaaccctgagaaagctcatccgtgaccctcgacatgcctaggtacttgcctaggttaaagccaggggacggattaagtctaaagacaattgggctgacagccagcaaccagccttagggtgtgaagggggatatccaacctttcacctagttggctgtgggtagccacagatgtcaagcagggtgagagaataggcatgaactca

SEQ ID NO: 12 - DNA, 23 bp, Homo sapiens
  hANGPTL4ClonF2 (1)..(23): human ANGPTL4 intron 3 forward primer
ctcgcggttctctaagttcacgg

SEQ ID NO: 13 - DNA, 24 bp, Homo sapiens
  hANGPTL4ClonR2 (1)..(23): human ANGPTL4 intron 3 reverse primer
aggaggcagggttgaggaaagaaa

SEQ ID NO: 14 - DNA, 24 bp, Mus musculus
  mANGPTL4ClonF2 (1)..(24): mouse ANGPTL4 intron 3 forward primer
cgttcacccttcttgacatctgtg

SEQ ID NO: 15 - DNA, 23 bp, Mus musculus
  mANGPTL4ClonR2 (1)..(23): mouse ANGPTL4 intron 3 reverse primer
cttggccttgacaagtcctctaa

SEQ ID NO: 16 - DNA, 711 bp, Homo sapiens
  cloned hANGPTL4 control region (1)..(711)
ctcgcggttctctaagttcacggccccacacggtttctctgtggtcctcatccttccctgcatctgtggctgtccacagccaactgggtgaaagtttggatccccctctcacaccctagggtcagtggcaggcttccagcactgtagacctgagggttctctcctccccaagctcccgctccttcccacctccgtgctgcccgcccccaaccccgccaggctagcatctcagcgtggtcagggtcctgtccaccctcccaaagccaccatcccaggatgaggggcttctggagggtgacgggggaaggcacaagtcctggctgggaaatgcggtggaagggggcagggttctgtgggttcgggactcccagactcttggctcaggcccgccaagtaggagaaagttcagagctgggaaggcgaacagctggcattcatggaagccacactggtggtttggccgcgtgcccatccttactggatgggaggaaagtaggggaaaggggagatgcctgaggggccggaaagcgtcttcctggtcactctgggcccgcccccacccccaccgtgcagactcatttcgacctttcccctacttttccggctgggctgggggcggttcctcccagtctggagcgtctgagcctccagacgtgctcaacgccatcctcccctcctccctccctctttctttcctcaaccctgcctcct

SEQ ID NO: 17 - DNA, 582 bp, Mus musculus
  cloned mANGPTL4 control region (1)..(582)
cgttcacccttcttgacatctgtggctctccacagccaactaggtgaaagtttggatatccccttcacaccctaaggctgcttgctggctttcagcccaatcctccttagacttaattcctcccctggcttttacttaggcaagtacctaggcatggcgagggtcatggatgagcttttccagggtcggcaggcataggttctggatgggtgatgtggtggaagagaagagctgagggggtggttttgtggctttggaactcccagactcagcctagccaagtagaggaaagttcagagctggagagacaaacagctggccctaatgggaaccacatgggtggtttggcagtgtgcctgtcccgattggatgagaggaaagtaggggaaagggagctagctgcccgagggattggaaagtatacttcccatctttctgggtctgcccccactcctggcagtgcagacggagctggctggcagaaagtgcctgcatgcccctcccccagtgacctttctcctactttccctggctgggctgggggtggttcttcctgaatttagaggacttgtcaaggccaag

SEQ ID NO: 18 - DNA, 28 bp, Mus musculus
  mANGPTL4 PPRE1 mutagenesis primer (1)..(28): Primer for deletion of 13 core DR1-encoding nucleotides of PPRE1 of mouse ANGPTL4
ctccacagccaactgatatccccttcac

SEQ ID NO: 19 - DNA, 28 bp, Mus musculus
  mANGPTL4 PPRE2 mutagenesis primer (1)..(28): Primer for deletion of 13 core DR1-encoding nucleotides of PPRE2 of mouse ANPTL4
cagcctagccaagtgagctggagagaca

SEQ ID NO: 20 - DNA, 28 bp, Mus musculus
  mANGPTL4 PPRE3 mutagenesis primer (1)..(28): primer for deletion of 13 core DR1-encoding nucleotides of PPRE3 of mouse ANGPTL4
gatgagaggaaagtctagctgcccgagg

SEQ ID NO: 21 - DNA, 28 bp, Mus musculus
  mANGPTL4 PPRE4 mutagenesis primer (1)..(28): primer for deletion of 13 core DR1-encoding nucleotides of PPRE4 of mouse ANGPTL4
tgcccctcccccagactttccctggctg

SEQ ID NO: 22 - DNA, 22 bp, Mus musculus
  mANGPTL4-PPRE 1 (1)..(22)
gccaactaggtgaaagtttgga

SEQ ID NO: 23 - DNA, 22 bp, Rattus norvegicus
  rANGPTL4-PPRE 1 (1)..(22)
gccaactaggtgaaaggttgga

SEQ ID NO: 24 - DNA, 22 bp, Canis familiaris
  dANGPTL4-PPRE 1 (1)..(22)
accaactgggtgaaagttcgga

SEQ ID NO: 25 - DNA, 22 bp, Homo sapiens
  hANGPTL4-PPRE 1 (1)..(22)
gccaactgggtgaaagtttgga

SEQ ID NO: 26 - DNA, 22 bp, Mus musculus
  mANGPTL4-PPRE 2 (1)..(22)
gccaagtagaggaaagttcaga

SEQ ID NO: 27 - DNA, 22 bp, Rattus norvegicus
  rANGPTL4-PPRE 2 (1)..(22)
gccaagtagaggaaagttcaga

SEQ ID NO: 28 - DNA, 22 bp, Canis familiaris
  dANGPTL4-PPRE 2 (1)..(22)
gccaagtaggagaagggtcaga

SEQ ID NO: 29 - DNA, 22 bp, Homo sapiens
  hANGPTL4-PPRE 2 (1)..(22)
gccaagtaggagaaagttcaga

SEQ ID NO: 30 - DNA, 22 bp, Mus musculus
  mANGPTL4-PPRE 3 (1)..(22)
ggaaagtaggggaaagggagct

SEQ ID NO: 31 - DNA, 22 bp, Rattus norvegicus
  rANGPTL4-PPRE 3 (1)..(22)
ggaaagtaggggaaagggagct

SEQ ID NO: 32 - DNA, 22 bp, Canis familiaris
  dANGPTL4-PPRE 3 (1)..(22)
ggaaagtaggggaaaggggagc

SEQ ID NO: 33 - DNA, 22 bp, Homo sapiens
  hANGPTL4-PPRE 3 (1)..(22)
ggaaagtaggggaaaggggaga

SEQ ID NO: 34 - DNA, 22 bp, Mus musculus
  mANGPTL4-PPRE 4 (1)..(22)
ggaaagtaggagaaaggtcact

SEQ ID NO: 35 - DNA, 22 bp, Rattus norvegicus
  rANGPTL4-PPRE 4 (1)..(22)
ggaaagtaggagaaaggtcact

SEQ ID NO: 36 - DNA, 22 bp, Canis familiaris
  dANGPTL4-PPRE 4 (1)..(22)
ggaaagtattggaaaggtcgcc

SEQ ID NO: 37 - DNA, 22 bp, Homo sapiens
  hANGPTL4-PPRE 4 (1)..(22)
gaaaagtaggggaaaggtcgaa

SEQ ID NO: 38 - DNA, 17 bp, Artificial
  Description of artificial sequence Consensus sequence of PPRE sequences of mouse, rat, dog and human
aactaggncaaaggtca

SEQ ID NO: 39 - DNA, 96 bp, herpes simplex virus 7
  minimal promoter region from the herpes simplex thymidine kinase gene (pTK37) (1)..(96)
gaattcggtccacttcgcatattaaggtgacgcgtgtggcctcgaacaccgagcgaccctgcagcgacccgcttaacagcgtcaacagcgtgccgc